(19) United States
(12) Patent Application Publication
(10) Pub. No.: US 2020/0102256 A1
(43) Pub. Date: Apr. 2, 2020

(54) Oligomerization Process

(71) Applicants: Emiel de Smit, Brussels (BE); Andrew D. Wiersum, Baton Rouge, LA (US); Michael W. Weber, Houston, TX (US); Marianne F. M. Smits, Mortsel (BE); Mechilium J. G. Janssen, Kessel-Lo, Leuven (BE); Marc P.H. Puttemans, Puttemans (BE); Larry L. laccino, Seabrook, TX (US)

(72) Inventors: Emiel de Smit, Brussels (BE); Andrew D. Wiersum, Baton Rouge, LA (US); Michael W. Weber, Houston, TX (US); Marianne F. M. Smits, Mortsel (BE); Mechilium J. G. Janssen, Kessel-Lo, Leuven (BE); Marc P.H. Puttemans, Puttemans (BE); Larry L. laccino, Seabrook, TX (US)

(21) Appl. No.: 16/493,609

(22) PCT Filed: Jan. 26, 2018
(86) PCT No.: PCT/EP2018/051982

§ 371 Date: Sep. 12, 2019

Related U.S. Application Data
(60) Provisional application No. 62/471,679, filed on Mar. 15, 2017.

(30) Foreign Application Priority Data
Apr. 27, 2017 (EP) 17168355.0

(51) Int. Cl. C07C 2/12

(52) U.S. Cl. CPC C07C 2/12; C07C 2529/40; C07C 2529/65; C07C 2529/18

(57) ABSTRACT

A process for oligomerizing an olefin feedstock to produce an olgiomerization product, a method for analysing an oligomerization product, and an oligomerization product are disclosed. Preferably, the process comprises contacting the olefin feedstock with an oligomerization catalyst under effective oligomerization conditions, wherein the olefin feedstock comprises at least 50 wt % of one or more C₆ olefins, based on the weight of the olefins in the olefin feedstock, and wherein the oligomerization catalyst comprises a crystalline molecular sieve, such as an intermediate pore size crystalline molecular sieve or a large pore size crystalline molecular sieve.

### FIELD OF THE INVENTION

The present invention concerns a method for oligomerizing an olefin feedstock to form an oligomerization product, an oligomerization product so produced, and a method of analysing an oligomerization product. More particularly, but not exclusively, this invention concerns a method for oligomerizing an olefin feedstock comprising at least 50 wt % of one or more C₆ olefins, based on the weight of the olefins in the olefin feedstock.

### BACKGROUND OF THE INVENTION

Many chemical processes take advantage of the reactivity of a carbon-carbon double bond to combine smaller olefins into larger molecules for use as fuels or intermediate feeds to other chemical processes. In such systems, a feed stream is typically passed through a reaction zone in which the olefin is contacted with a catalyst. The catalyst enables a chemical reaction in which the olefin molecules combine into larger molecules.

In typical oligomerization processes, process parameters can have a significant impact on the product quality. For example, the feed material, the catalyst, and/or the process conditions often impact the size and shape of the molecules in the product. Oligomerization processes generally produce a distribution of molecule sizes and, it is often desirable to make that size distribution as narrow as possible. Oligomerization processes also generally produce a distribution of molecule isomers, for example varying from linear shaped molecules to molecules having a more branched structure, and varying from alpha olefins (where a carbon-carbon double bond is located between the first and second carbon atoms of an oligomer chain) to olefins in which the carbon-carbon double bond is located further along the oligomer chain. The distribution of molecular sizes, shapes and carbon-carbon double-bond locations in an oligomerization product are important product characteristics, for example because product size distribution and double bond location can affect the ability of the product to be used as a feed in subsequent chemical processes and the performance of final consumer products made from such feedstocks.

C₁₂ olefins (dodecene) are a particularly useful oligomerization product, for example because C₁₂ olefins are useful intermediates in the manufacturing of a wide variety of products, including plasticizers (such as ditridecyl phthalate, DTDP), surfactants and lubricants. In the manufacture of such products, the C₁₂ olefin may, for example, be converted to another intermediate, such as a C₁₃ alcohol (tridecyl alcohol, TDA). Typically, C₁₂ oligomers are made by the catalytic oligomerization of feed streams comprising C₃ and/or C₄ olefins (propylene and/or butylene), which often produces complex mixtures of mainly C₁₂ (dodecene) isomers, but also containing some C₁₁ (undecene) and C₁₃ (tridecene) isomers. Such oligomerization processes are disclosed in, for example, U.S. Pat. No. 8,598,396 (which discloses a process for oligomerizing olefins over a phosphoric acid catalyst, sPa), U.S. Pat. No. 4,814,540 (which discloses a process for oligomerizing propylene isomers over a catalyst made up of a transition metal and an organometallic compound) and U.S. Pat. No. 3,932,553 (which discloses a process for oligomerizing propylene isomers over a boron trifluoride catalyst).

In some cases, process conditions can be adjusted to improve control of product distribution in oligomerization processes. The temperature, composition, flow rate, catalyst type, and amount of catalyst in the reaction zone are examples of parameters that can be adjusted to control selectivity for the desired product in a given reaction zone.

In some processes, selecting conditions that provide too high a rate of conversion of starting material olefins in the feed stream can result in the formation of a significant quantity of unwanted heavy by-products. For example, it may be that it is desirable to find a balance between maximizing the conversion of olefin starting material into the oligomer product and minimizing the production of heavy by-products. Accordingly, process parameters are often selected that avoid 100% conversion of the starting material olefins in the feed stream. To improve overall process efficiency, un-reacted starting material olefins can, for example, be separated from the product stream and recycled back to the reactor to increase the total fraction of feed that is converted to product. U.S. Pat. No. 7,678,953 describes an oligomerization system in which the product is separated into light olefin stream that is recycled to the reactor and a first hydrocarbon product. Typically, such a recycle stream is purged prior to reintroduction into the reactor to avoid the build-up of light hydrocarbons in the system. It will be appreciated that when the rate of conversion of starting material olefins in the feed stream is too low, more of the starting material olefins may be lost in the purging of the recycle, and so there may be an optimum per-pass conversion that maximizes product yield and minimizes loss to heavy by-products and purging. In addition to increasing overall conversion, the recycle can also be used to control conditions in the reaction zone, for example because the recycle stream effectively acts as a feed stream to the reactor. U.S. Pat. No. 6,080,903 describes an olefin oligomerization process in which a non-reactive component is added to the recycle stream to improve catalyst life.

In some cases, control of parameters and conditions of a reaction zone can be improved by separating the reaction zone into separate reactor vessels. For example, heat exchangers can be used to remove the heat of reaction between adjacent reactors to approach isothermal reacting conditions. U.S. Pat. No. 7,588,738 describes systems in which multiple reactors are employed in an olefin oligomerization process, and in which different catalysts are employed in each reactor and temperature is independently controlled to optimize the conditions with respect to each catalyst. Another benefit is that multiple reactors can, for example, allow for partial shutdown of the reaction zone, thus adding some redundancy the reaction system (e.g. because a single reactor can be shut down for maintenance without shutting down the entire system). Such capability may be especially useful if the catalyst loses activity as it ages. Furthermore, the use of multiple reactors may, for example, allow for the catalyst to be replaced or regenerated one reactor at a time.

The properties of C₁₁, C₁₂ and C₁₃ olefins are very different, and as such tend to be more or less suitable for a broad variety of end applications, for example including the production of TDA. Despite their different properties, the distillation cut points for C₁₁, C₁₂ and C₁₃ olefins are very similar, making the separation of olefin mixtures challenging. Furthermore, it has been found that less branched C₁₂ olefins are converted to TDA more easily (it is believed that the more highly branched C₁₂ isomers are less reactive in conversion to TDA than less branched isomers). Thus, it is desirable to maximize the selectivity for a particular group of olefins (e.g. C12 olefins) and/or to maximize the selectivity for one or more isomers (e.g. linear olefins and/or alpha-olefins).

It can also be difficult to analyze the chemical composition of C₁₁/C₁₂/C₁₃ olefin mixtures, for example because of strong overlaps between the C₁₁/C₁₂/C₁₃ isomers in chromatographic measurements and because of the large number of different isomers in each olefin group. In particular, conventional gas chromatography typically does not allow the identification of the branchiness and olefin type (e.g. alpha-olefin or otherwise) of individual components of a C₁₁/C₁₂/C₁₃ mixture. Similarly, Nuclear Magnetic Resonance (NMR) spectroscopy is typically only able to measure the average branchiness of an entire C₁₁/C₁₂/C₁₃ mixture, and not, for example, the branchiness of the C₁₂ fraction within the mixture.

There remains a need for providing improved routes to C₁₂ olefins that provide C₁₂ products comprising a lower proportion of unwanted C₁₁ and C₁₃ olefins and/or that provide less branched C₁₂ products. In other words, there is a need for an oligomerization process for producing C₁₂ olefins having a narrower carbon number distribution (CND) and/or a lower average branchiness. There also remains a need for an analytical method capable of better identifying the oligomeric and isomeric makeup of an olefin mixture, such as a C₁₁/C₁₂/C₁₃ mixture.

### SUMMARY OF THE INVENTION

The present invention provides, according to a first aspect, a process for oligomerizing an olefin feedstock to form an oligomerization product, wherein the process comprises contacting the olefin feedstock with an oligomerization catalyst under effective oligomerization conditions; wherein, the olefin feedstock comprises at least 50 wt % of one or more C₆ olefins, based on the weight of the olefins in the olefin feedstock; and wherein, the oligomerization catalyst comprises a crystalline molecular sieve, such as an intermediate pore size crystalline molecular sieve or a large pore size crystalline molecular sieve.

The present inventors have surprisingly found that using an olefin feedstock comprising at least 50 wt % of one or more C₆ olefins in a crystalline molecular sieve-catalysed oligomerization reaction provides a unique and improved route to olefin oligomers, and more particularly to C₁₂ olefin oligomers. In comparison to a typical oligomerization process utilizing a feed comprising C₃ olefins (e.g. a propylene feed), the present inventors have found that the process of the present invention is more selective for the production of C₁₂ rather than C₁₁/C₁₃ oligomers, and is more selective for the production of less branched rather than more highly branched isomers. Furthermore, an oligomerization product produced from a C₆ olefin feed in a process according to the first aspect of the invention may, for example, facilitate the production of TDA having a narrower CND and lower branchiness than TDA produced from an oligomerization product made by a C₃ olefin oligomerization process. As a result, overall process yield from olefin starting material to useful TDA product may be improved. Without wishing to be bound by theory, the present inventors believe that the process of the first aspect of the invention provides advantages in terms of selectivity and yield at each stage of the process leading from olefin feed to TDA product, as compared to a process starting from a C₃ olefin feed. FIG. 1 shows a set of graphs comparing the product distribution at various stages of producing an oligomerization product (and an alcohol product produced therefrom) from a C₃ feed and from a C₆ olefin feed. In FIG. 1, the sequence of graphs under heading A show product distribution at each stage of a process using a C₃ olefin feed, and the sequence of graphs under heading B show product distribution at each stage of a process using a C₆ olefin feed. Stage 1010 is the olefin oligomerization stage, stage 1020 is the oligomerization product fractionation stage, stage 1030 is the oligomer to TDA conversion stage, and stage 1040 is the TDA fractionation stage that yields the final TDA product. In stage 1010, the oligomerization product produced from a C₃ olefin feed comprises mainly C₁₂ olefins (indicated by curve 1011), but also comprises significant amounts of C₁₁ olefins (indicated by curve 1012) and C₁₃ olefins (indicated by curve 1013), giving a broad CND. The fractionation cut points for separating the C₁₂ product from the C₁₁ and C₁₃ by-products are shown by dashed lines 1014a and 1014b. In contrast, in stage 1010, the oligomerization product produced from a C₆ olefin feed comprises mainly C₁₂ olefins (indicated by curve 1011) and a much smaller proportion of C₁₁ olefins (indicated by curve 1012) and C₁₃ olefins (indicated by curve 1013), giving a narrower CND. Furthermore, the distribution of C₁₂ isomers in the C₆ oligomerization product is weighted towards more linear isomers—the more branched isomers make up the left-hand side of C₁₂ oligomer product distribution curve 1011 (for comparison, the position of the C₁₂ oligomer product distribution curve for the C₃ olefin oligomerization product is shown next to that of the C₆ olefin oligomerization product by dotted line 1015 in FIG. 1). At stage 1020, which shows product distribution after fractionation of the oligomerization product, FIG. 1 shows that a significant proportion of the C₃ olefin oligomerization product has been lost during fractionation, and that while the C₁₂ oligomers (indicated by curve 1021) are still the major component (60%) of the fractionated oligomerization product, there is still a significant quantity of the C₁₁ (indicated by curve 1022, 30%) and C₁₃ (indicated by curve 1023, 10%) by-products. In contrast, at stage 1020, a much smaller proportion of the C₆ olefin oligomerization product has been lost during fractionation, and the fractionated oligomerization product comprises considerably less C₁₁ (indicated by curve 1022) and C₁₃ (indicated by curve 1023) by-products as compared to the C₁₂ oligomers (indicated by curve 1021). At stage 1030, the major product of olefin to alcohol conversion is the desired C₁₃ alcohol (indicated by curve 1031a), although there are significant amounts of C₁₂ alcohol (indicated by curve 1032a) and C₁₄ alcohol (indicated by curve 1033a) by-product. Furthermore, a substantial amount of C₁₂ olefin is unconverted (indicated by curve 1031b), as well as substantial amounts of C₁₁ (indicated by curve 1032b) and C₁₃ (indicated by curve 1033b) being left unconverted (in total, 30% of the fractionated C₃ olefin oligomerization product is unconverted). In contrast, at stage 1030, not only does the desired C₁₃ alcohol make up a higher proportion of the olefin to alcohol conversion product produced from the fractionated C₆ olefin oligomer (compare relative amounts of C₁₃ alcohol, indicated by curve 1031a, with the amounts of C₁₂ alcohol, indicated by curve 1032a, and C₁₄ alcohol, indicated by curve 1033a, at stage 1030 in FIG. 1), the amount of unconverted C₁₁ (curve 1032b), C₁₂ (curve 1031b) and C₁₃ (curve 1033b) olefins is lower. Without wishing to be bound by theory, it is believed that the higher proportion of higher reactivity linear oligomers in the C₆ olefin oligomerization product as compared to the C₃ olefin oligomerization product provides an improvement in conversion to TDA. The fractionation cut points for isolating TDA from the stage 1030 mixture are indicated by dashed lines 1034a and 1034b. At stage 1040, a substantial amount of the TDA produced by the C₃ olefin oligomerization process (around 14%) is lost during fractionation due to the broad boiling point range of the TDA product. In contrast, at stage 1040, the narrower CND of the TDA produced by the C₆ olefin oligomerization process is believed to result in a lower loss of product during fractionation. Furthermore, at stage 1040, in the TDA produced from the C₃ olefin oligomerisation product, while the C₁₃ TDA (indicated by curve 1031a) is still the major component of the TDA product, there is still a significant quantity of the C₁₂ (indicated by curve 1032a) and C₁₄ (indicated by curve 1033a) TDA by-products. In contrast, for the TDA produced from the C₆ olefin oligomerization product, the relative proportion of C₁₃ TDA (indicated by curve 1031a) is considerably higher than that of the C₁₂ (indicated by curve 1032a) and C₁₄ (indicated by curve 1033a) TDA by-products.

According to a second aspect, the present invention provides a process for oligomerizing an olefin feedstock to form an oligomerization product, wherein the process comprises: contacting the olefin feedstock with an oligomerization catalyst in a reaction zone under effective oligomerization conditions, the oligomerization catalyst comprising a crystalline molecular sieve, wherein the reaction zone comprises a plurality of reactors arranged in series, each reactor housing a portion of the oligomerization catalyst; the process being operated in a first configuration, i.e. under first oligomerization conditions, for a first operating period and subsequently in a second configuration, i.e. under second oligomerization conditions, for a second operating period, and wherein the outlet temperature of the last reactor in the series of multiple reactors is substantially the same in the first and second configurations, and the inlet and/or outlet temperature of at least one of the reactors other than the last reactor, or the inlet temperature of the last reactor, in the second configuration differs from the corresponding inlet and/or outlet temperature of at least one of the reactors other than the last reactor, or the inlet temperature of the last reactor, in the first configuration. Preferably, the inlet and/or outlet temperature of at least one of the reactors other than the last reactor, or the inlet temperature of the last reactor, in the first configuration is lower than the corresponding inlet and/or outlet temperature of at least one of the reactors other than the last reactor, or the inlet temperature of the last reactor, in the second configuration. Optionally, the olefin feedstock comprises at least 50 wt % of one or more C₆ olefins, based on the weight of the olefins in the olefin feedstock. Optionally, the oligomerization catalyst comprises an intermediate pore size crystalline molecular sieve or a large pore size crystalline molecular sieve.

According to the second aspect of the invention, if, for example: the reaction zone comprises two reactors arranged in series and, in the first configuration, the inlet temperature of the first reactor is TI₁₋₁, the inlet temperature of the second reactor is TI₂₋₁, the outlet temperature of the first reactor is TO₁₋₁ and the outlet temperature of the second reactor is TO₂₋₁, and, in the second configuration, the inlet temperature of the first reactor is TI₁₋₂, the inlet temperature of the second reactor is TI₂₋₂, the outlet temperature of the first reactor is TO₁₋₂ and the outlet temperature of the second reactor is TO₂₋₂; then, TO₂₋₁ is substantially equal to TO₂₋₂, and: TI₁₋₁ is different to TI₁₋₂, TI₂₋₁ is different to TI₂₋₂, and/or TO₁₋₁ is different to TO₁₋₂. Preferably, TI₁₋₁ is lower than TI₁₋₂, TI₂₋₁ is lower than TI₂₋₂, and/or TO₁₋₁ is lower than TO₁₋₂.

The present inventors have surprisingly found that, by using multiple reactors, by fixing the outlet temperature of the last reactor, and by varying reactor inlet and/or outlet temperatures upstream of the outlet of the last reactor, reactor temperatures can be tailored to catalyst condition without altering product oligomer properties (for example, without altering the isomer distribution in the product oligomer). Without wishing to be bound by theory, it is believed that increasing reactor temperature can compensate for reductions in yield resulting from catalyst aging, and that fixing the outlet temperature of the last reactor avoids variation in product properties due to such temperature changes. In other words, the present inventors have found that the process of the second aspect of the invention allows product yield and product properties to be kept substantially constant when the process is operated over an extended period.

According to a third aspect, the present invention provides a method of analysing a hydrocarbon mixture by chromatography-mass spectrometry, the hydrocarbon mixture comprising a plurality of Cₙ olefin isomers and a plurality of Cₙ₊₁ olefin isomers, wherein n is from 8 to 18, preferably 11 or 12, the method comprising: selecting a Cₙ molecular ion, such as a C₁₂ molecular ion, for example a C₁₂ olefin molecular ion having an m/z of 168, for mass spectrometry detection; selecting a chromatography start point and a chromatography end point to define a chromatography retention time zone extending from the start point to the end point; dividing the chromatography retention time zone into a plurality of sections, such as at least three sections, for example at least three equally sized sections, each section corresponding to a group of molecular ion isomers; and, determining total detection of the molecular ion in each of the plurality of retention time zone sections thereby determining the relative amounts of each group of molecular ion isomers. It will be appreciated that the particular molecular ion selected for the analysis method is likely to vary according to either or both of the Cₙ hydrocarbon of interest and the mass spectrometry method/equipment. For example, it may be that a molecular ion of 168 m/z is selected for the direct analysis of a C₁₂ olefin isomer mixture (168 m/z corresponding to C₁₂H₂₄), or that a molecular ion of 170 m/z is selected for the analysis of a hydrogenated C₁₂ olefin isomer mixture (170 m/z corresponding to C₁₂H₂₆).

It may be that, for example, one of the retention time zones includes the retention time of the most branched Cₙ olefin isomer, and one of the other retention time zones includes the retention time of the most linear Cₙ olefin isomer. Optionally, the analysis method of the third aspect of the invention is used to analyse the oligomerization product produced by the process of the first or second aspects of the invention. It will be appreciated that the method of the third aspect of the invention may, for example, comprise mass spectrometry coupled with any separation technique by with molecules are separable based on their physical properties, e.g. some form of chromatography, such as gas chromatography.

The present inventors have unexpectedly found that the analysis method of the third aspect of the invention provides a particularly reliable and easily repeatable method of determining the relative proportions of, for example, highly linear, medium branched and highly branched isomers of a Cₙ olefin in a mixture of Cₙ and Cₙ₊₁ olefins. Without wishing to be bound by theory, the present inventors believe that the branchiness of an olefin isomer influences the retention time of that isomer in chromatography, and that by coupling the chromatographic analysis method with mass spectroscopy, Cₙ isomers can also be separated from Cₙ₊₁ isomers. The present inventors also believe that grouping molecular ion isomers into three or more groups, rather than attempting to identify each individual isomer, makes the analysis method particularly suitable for use with complex mixtures of isomers, such as those produced in catalytic oligomerization processes. Furthermore, the present inventors have found that the analysis method of the third aspect of the invention is particularly effective in allowing a comparison between different catalytic oligomerization product samples, such as those prepared by different types of catalytic oligomerization process.

According to a fourth aspect, the present invention provides an olefin composition comprising from 70 to 95 wt % C₁₂ olefin isomers, based on the weight of the olefin composition, wherein the olefin composition comprises at least 50 mol % olefin isomers of type II and IVA, based on the moles of the olefin isomers in the olefin composition, and wherein the average branchiness of the olefin composition is in the range of from 2.6 to 3.3, optionally 2.6 to 2.95, for example 2.85 to 2.95.

According to a fifth aspect, the present invention provides an olefin composition having an initial boiling point of 185° C. and a final boiling point of 210° C. and comprising from 70 wt % to 95 wt % C₁₂ olefin isomers, from 8 wt % to 20 wt % C₁₁ olefins, and from 1 wt % to 12 wt % C₁₃ olefins, based on the weight of the olefin composition. Preferably said composition comprises at least 50 mol % olefin isomers of type II and IVA, based on the moles of the olefin isomers in the olefin composition, and wherein the average branchiness of the olefin composition is in the range of from 2.6 to 3.3, optionally 2.6 to 2.95, for example 2.85 to 2.95.

According to a sixth aspect, the present invention provides an olefin composition comprising from 70 to 95 wt % C₁₂ olefin isomers having a chromatography retention time falling in a retention time zone extending from the retention time of heptane, 2, 2, 6, 6-tetramethyl-4-methylene to the retention time of 1-dodecene, based on the weight of the olefin composition, the retention times of heptane, 2, 2, 6, 6-tetramethyl-4-methylene, 1-dodecene, and the C₁₂ olefin isomers of the olefin composition being measured on a chromatography column configured to separate molecules by boiling point and/or branchiness, and the retention time zone being divided into at least three equally sized retention time sub-zones including sub-zones A, B and C, sub-zone A including the retention time of heptane, 2, 2, 6, 6-tetramethyl-4-methylene and sub-zone C including the retention time of 1-decene, and wherein from 3 wt % to 15 wt % of said C₁₂ olefin isomers have a retention time falling in sub-zone A, from 45 wt % to 70 wt % of said C₁₂ olefin isomers have a retention time falling in sub-zone B, and from 15 wt % to 50 wt % of said C₁₂ olefin isomers have a retention time falling in sub-zone C.

The present inventors have found that the unique olefin compositions of the fourth, fifth and sixth aspects of the present invention are particularly useful as intermediates in, for example, the preparation of TDA and the products produced therefrom. Without wishing to be bound by theory, the present inventors believe that the compositions can be prepared reliably and in a convenient manner from readily available starting materials and have a particularly useful makeup of C₁₂ olefin isomers suitable for forming useful TDA compositions.

It will of course be appreciated that features described in relation to one aspect of the present invention may be incorporated into other aspects of the present invention. For example, it will be appreciated that the olefin composition of the fourth or fifth aspects of the invention may be prepared by the method of the first or second aspects of the invention, and/or analysed by the analysis method of the third aspect of the invention, and vice versa.

### DESCRIPTION OF THE FIGURES

FIG. 1 shows a set of graphs comparing the product distribution at various stages of producing an oligomerization product (and an alcohol product produced therefrom) from a C₃ feed and from a C₆ olefin feed;

FIG. 2 shows a graph plotting process temperature against oligomerization product branchiness determined by NMR for an oligomerization product produced according to the process according to the first aspect of the invention;

FIG. 3 shows a graph plotting process temperature against oligomerization product quaternary carbon content for the oligomerization product produced for the analysis of FIG. 2;

FIG. 4 shows a further graph plotting process temperature against oligomerization product quaternary carbon content for an oligomerization product produced according to the process according to the first aspect of the invention;

FIG. 5 shows a graph plotting oligomerization product quaternary carbon content against oligomerization product branchiness for the oligomerization product produced for the analysis of FIG. 2;

FIG. 6 shows graphs plotting process temperature against oligomerization product carbon number distribution against temperature for the oligomerization product produced for the analysis of FIG. 2;

FIG. 7 shows a graph plotting carbon number against simulated mass fraction for an oligomerization product produced from a C₃ olefin feed and from a C₆ olefin feed;

FIG. 8 shows a process schematic for an oligomerization process according to the first aspect of the invention;

FIG. 9 shows a pair of graphs showing variation of product yield and per pass conversion with recycle ratio, and variation of loss to purge, saturates concentration and loss to heavies with recycle ratio, while overall conversion is kept constant, in an oligomerization process according to the first aspect of the invention;

FIG. 10 shows a pair of graphs showing variation of product yield and overall conversion with recycle ratio, and variation of loss to purge, saturates concentration and loss to heavies with recycle ratio, while per pass conversion is kept constant, in an oligomerization process according to the first aspect of the invention;

FIG. 11 shows a graph plotting process temperature against oligomerization product quaternary carbon content and branchiness for an oligomerization product produced by a process according to the second aspect of the invention;

FIG. 12 shows a process schematic for an oligomerization process according to the second aspect of the invention;

FIG. 13 shows a full scan GC-MS chromatogram of a C₁₁, C₁₂, C₁₃ olefin mixture;

FIG. 14 shows an overlay of three extracted ion GC-MS chromatograms of C₁₁ olefins (154 m/z), C₁₂ olefins (168 m/z) and C₁₃ olefins (182 m/z);

FIG. 15 shows an overlay of two extracted ion GC-MS chromatograms of C₁₂ olefins (168 m/z) from two different plant samples;

FIG. 16 depicts branching indexes for C₁₂ olefins from various sources, the branching indexes being determined by an analysis method according to the third aspect of the invention; and,

FIG. 17 depicts branching indexes for C₁₂ olefins from various sources (including plant samples and samples prepared by a process according to the first aspect of the invention), the branching indexes being determined by an analysis method according to the third aspect of the invention.

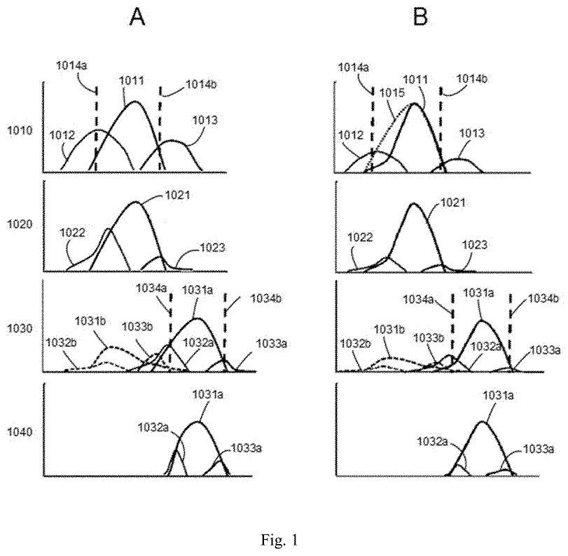

### DETAILED DESCRIPTION OF THE INVENTION

As used herein, an intermediate pore size crystalline molecular sieve is a crystalline molecular sieve having a pore size of from 5 Å to 7 Å, and a large pore size crystalline molecular sieve is a crystalline molecular sieve having a pore size greater than 7 Å. Such crystalline molecular sieves/zeolites are described in “Atlas of Zeolite Structure Types”, eds. W. H. Meier and D. H. Olson, Butterworth-Heineman, Third Edition, 1992, which is hereby incorporated by reference.

It will be appreciated that when a multiplicity of reactors is described as being ‘arranged in series’, such reactors are arranged sequentially such that the effluent from one reactor is passed as at least part of the feed to the next reactor in the series, and so on. It will also be appreciated that one or more other reactors, not forming part of the series, may be arranged in parallel to the series of reactors.

As used herein, a major portion of a feed or composition, for example, means more than 50 wt % of said feed or composition, and a minor portion means up to 50 wt % of said feed or composition.

Optionally, in the process of the first aspect of the invention, the crystalline molecular sieve comprises at least one of an intermediate pore size crystalline molecular sieve having 10-membered ring pores, or a large pore size crystalline molecular sieve having 12-membered ring pores. Preferably, the crystalline molecular sieve comprises an intermediate pore size molecular sieve having 10-membered ring pores. Optionally, when the crystalline molecular sieve comprises an intermediate pore size crystalline molecular sieve, the intermediate pore size crystalline molecular sieve is a zeolite having a structure type selected from the list consisting of AEL, MFI, MFS, MEL, MRE, MTW, MWW, EUO, MTT, HEU, FER, and TON. For example, it may be that the intermediate pore size crystalline molecular sieve is a zeolite selected from the list consisting of MCM-22, MCM-49, MCM-56, SAPO-11, ZSM-5, EMM-20, ZSM-11, ZSM-22, ZSM-23, ZSM-35, ZSM-48, ZSM-50 and ZSM-57, optionally from the list consisting of ZSM-5, ZSM-11, ZSM-48 and ZSM-57. Optionally, when the crystalline molecular sieve comprises a large pore size crystalline molecular sieve, the large pore size crystalline molecular sieve is a zeolite having a structure type selected from the list consisting of LTL, VFI, MAZ, MEI, FAU, EMT, OFF, BEA, and MOR. For example, it may be that the large pore size crystalline molecular sieve, is a zeolite selected from the list consisting of Mordenite, Beta and Ultrastable Y (USY).

Optionally, the olefin feedstock comprises at least 60 wt %, for example at least 70 wt %, such as at least 80 wt %, of one or more C₆ olefins, based on the weight of the olefins in the olefin feedstock. Preferably, the olefin feedstock comprises at least 55 wt % of one or more C₆ olefins, based on the weight of the olefin feedstock, optionally wherein the olefin feedstock comprises 55 wt % to 65 wt % of one or more C₆ olefins, based on the weight of the olefin feedstock. Optionally, at least a portion of the olefin feedstock, such as a major portion of the olefin feedstock is a stream recovered from a product stream from a light olefin oligomerization process, such as a process for oligomerizing C₂, C₃, C₄, and/or C₅ olefins. It may be that, for example, the product stream is a by-product stream. Additionally or alternatively, it may be that at least a portion of the olefin feedstock, such as a major portion of the olefin feedstock is a stream recovered from the product of a thermal hydrocarbon conversion process, such as steam cracking or steam coking. It may be that, for example, at least a portion of the olefin feedstock, such as a major portion of the olefin feedstock is a stream recovered from the product of a heavy hydrocarbon catalytic conversion process, such as a fluidized catalytic cracking (FCC) process. Optionally, at least a portion of the olefin feedstock, such as a major portion of the olefin feedstock is a stream recovered from the product of a methanol catalytic conversion process, such as methanol-to-olefins, methanol-to-propylene, methanol-to-aromatics or methanol-to-gasoline catalytic conversion process. Additionally or alternatively, it may be that at least a portion of the olefin feedstock, such as a major portion of the olefin feedstock is a stream recovered from the product of a syngas catalytic conversion process, such as a Fisher Tropsch, syngas-to-olefins, or syngas-to-aromatics process. It will be appreciated that the olefin feedstock may, for example, comprise any combination of streams recovered from said product streams. Suitable methods for recovery from said product streams include, for example, distillation, adsorption, extraction, membrane separation and combinations thereof.

Preferably, the oligomerization product produced by the process of the first aspect of the invention comprises C₁₂ olefins, preferably the oligomerization product comprises at least 60 wt % C₁₂ olefins, such as from 60 wt % to 95 wt % C₁₂ olefins, based on the weight of the olefins in the oligomerization product.

Optionally, the effective oligomerization conditions include at least one of, such as at least two of, for example all of: (i) a temperature of from 100° C. to 330° C., such as from 150° C. to 280° C., for example from 200° C. to 230° C.; (ii) a pressure of from 3 MPa to 10 MPa, such as from 4 MPa to 8 MPa, for example from 5 MPa to 6 MPa; and a weight hourly space velocity from 0.1 to 20 h⁻¹, such as from 0.5 to 12 h⁻¹, for example from 0.8 to 3 h⁻¹.

Optionally, the process comprises separating the oligomerization product into a recycle stream and a further processing stream, the recycle stream comprising olefins of carbon number less than 12 and the further processing stream comprising oligomers. Preferably, the process comprises contacting the olefin feedstock with the oligomerization catalyst under the effective oligomerization conditions in the presence of the recycle stream. Optionally, the process comprises separating the further processing stream into a product stream and a heavies stream, the product stream comprising oligomers, for example C₁₂ olefin, and the heavies stream comprising heavy by-products, for example one or more of C₆-trimers (e.g. C₁₈ olefins) and C₆-tetramers (e.g. C₂₄ olefins). Additionally or alternatively, the process may comprise further separating a purge stream from the recycle stream, the purge stream comprising low reactivity by-products. It may be that, for example, the purge stream is in the form of a slip stream (e.g. having the same composition as the recycle stream). It will be appreciated that the cut point used to separate the oligomerization product into a recycle stream and a further processing stream, and/or the cut point used to separate the further processing stream into a product stream and a heavies stream, will vary according to, for example, the processing equipment available, the pressure of the stream and the nature of the target oligomerization product. For example, it may be that the cut point for separating the oligomerization product into a recycle stream and a further processing stream is set between C₉ (e.g. nonene) and C₁₀ (e.g. decene), such as between the boiling points of C₉ and C₁₀ olefins). Additionally or alternatively, it may be that the cut point for separating the further processing stream into a product stream and a heavies stream is set between C₁₄ and C₁₅, such as between the boiling points of C₁₄ and C₁₅ olefins. It may be that, for example, such a recycle system improves overall % conversion of olefins in the starting material to oligomerization product.

Optionally, the process comprises operating the process in a first process configuration in which the recycle stream is recycled at a first recycle flow rate, the olefin feedstock is contacted with the oligomerization catalyst at a first temperature, and olefins in the olefin feedstock are converted to oligomers in the further processing stream at a first conversion rate; and, operating the process in a second process configuration in which the recycle stream is recycled at a second recycle flow rate, the olefin feedstock is contacted with the oligomerization catalyst at a second temperature, and olefins in the olefin feedstock are converted to oligomers in the further processing stream at a second conversion rate; wherein the second recycle flow rate is greater than the first recycle flow rate, and wherein the first temperature and the second temperature are selected such that the first conversion rate is substantially the same as the second conversion rate, optionally wherein the first conversion rate and the second conversion rate are between 65% and 85%, such as about 75%. It may be that, for example, the second temperature is higher than the first temperature. In other words, during continuous operation of the process, the reactor temperature is adjusted (e.g. increased) in order to maintain the overall conversion rate (i.e. rate of conversion of starting material olefins to product oligomers) at a substantially constant level while increasing the recycle flow rate. For example, it may be that the ratio of fresh feed to recycle feed flowing into the reaction zone varies from an initial ratio of 0 to a subsequent ratio of from 0.3 to 0.5 (such as about 0.34), and that the temperature at which the olefin feedstock is contacted with the oligomerization catalyst varies from an initial temperature to a subsequent temperature, wherein the subsequent temperature is from 2° C. to 10° C., such as 3° C. to 5° C., greater than the initial temperature (e.g. from an initial temperature of about 126° C. to a subsequent temperature of about 129° C.). The present inventors have found that such an arrangement reduces the amount of starting material olefins converted to unwanted heavy by-products.

Alternatively, the process is optionally operated in a first process configuration in which the recycle stream is recycled at a first recycle flow rate, the olefin feedstock is contacted with the oligomerization catalyst at a first temperature, and olefins comprising: a) olefins in the olefin feedstock, and b) olefins in the recycle stream, are converted to oligomers in the further processing stream at a first conversion rate; and, operating the process in a second process configuration in which the recycle stream is recycled at a second recycle flow rate, the olefin feedstock is contacted with the oligomerization catalyst at a second temperature, and olefins comprising: a) olefins in the olefin feedstock, and b) olefins in the recycle stream, are converted to oligomers in the further processing stream at a second conversion rate; wherein the second recycle flow rate is greater than the first recycle flow rate, and wherein the first temperature and the second temperature are selected such that the first conversion rate is substantially the same as the second conversion rate, optionally wherein the first conversion rate and the second conversion rate are between 65% and 85%, such as about 75%. It may be that, for example, the second temperature is higher than the first temperature. In other words, during continuous operation of the process, the reactor temperature is adjusted (e.g. increased) in order to maintain the ‘per pass conversion rate’ (i.e. rate of conversion of all olefins entering the reaction zone, including recycled and fresh olefins, to product oligomers) at a substantially constant level while increasing the recycle flow rate. For example, it may be that the ratio of fresh feed to recycle feed flowing into the reaction zone varies from an initial ratio of 0 to a subsequent ratio of from 0.2 to 0.4 (such as about 0.25), and that the temperature at which the olefin feedstock is contacted with the oligomerization catalyst varies from an initial temperature to a subsequent temperature, wherein the subsequent temperature is from 4° C. to 12° C., such as 5° C. to 7° C., greater than the initial temperature (e.g. from an initial temperature of about 126° C. to a subsequent temperature of about 132° C.). The present inventors have found that such an arrangement reduces the amount of starting material olefins lost during recycle, e.g. lost to the purge stream.

Preferably, the olefin feedstock is contacted with a first oligomerization catalyst under first effective oligomerization conditions in a first reactor to form a first effluent, and wherein the effluent is contacted with a second oligomerization catalyst in a second reactor under second effective oligomerization conditions to form a second effluent, the second oligomerization catalyst comprising a crystalline molecular sieve. The second oligomerization catalyst may be the same as or different from the first oligomerization catalyst. It will be appreciated that the crystalline molecular sieve of the second oligomerization catalyst may be any crystalline molecular sieve as described in relation to the crystalline molecular sieve of the oligomerization catalyst described herein. Optionally, the first and second effective oligomerization conditions include the conditions disclosed above in relation with the first aspect of the invention. It may be that, for example, the second effective oligomerization conditions include at least one of, such as at least two of, for example all of: (i) a temperature substantially the same as the temperature of the first effective oligomerization conditions; (ii) a pressure substantially the same as the first effective oligomerization conditions; (iii) a weight hourly space velocity substantially the same as the weight hour space velocity of the first effective oligomerization conditions. Additionally or alternatively, it may be that, for example, the second effective oligomerization conditions include at least one of, such as at least two of, for example all of: (i) a temperature different to the temperature of the first effective oligomerization conditions; (ii) a pressure different to the pressure of the first effective oligomerization conditions; (iii) a weight hourly space velocity different to the weight hour space velocity of the first effective oligomerization conditions. Optionally, the second effective oligomerization conditions include a temperature that differs from, such as differs by at least 20° C. from, for example differs by at least 40° C. from, the temperature of the first effective oligomerization conditions, optionally wherein the temperate of the second effective oligomerization conditions is higher than the temperature of the first effective oligomerization conditions. Preferably, the first effluent comprises C₁₂ olefins and the second effluent comprises C₁₂ olefins, and wherein the second effluent comprises a greater wt % C₁₂ olefins, such as at least 1 wt % more C₁₂ olefins, for example at least 2 wt % more C₁₂ olefins, based on the weight of the olefins in the second effluent, than the wt % C₁₂ olefins in the first effluent, based on the weight of the olefins in the first effluent. Optionally, the first oligomerization catalyst has been used in an oligomerization process for a first reaction period, and the second oligomerization catalyst has been used in an oligomerization process for a second reaction period, wherein the second reaction period is different to the first reaction period, optionally wherein the second reaction period is longer than the first reaction period. In other words, it may be that the second oligomerization catalyst is older (i.e. it has been used as a catalyst for a longer period of time) than the first oligomerization catalyst. It will be appreciated that a catalyst may, for example, be regenerated, thus starting a new reaction period for the regenerated catalyst.

Optionally, the second effluent is contacted with a third oligomerization catalyst in a third reactor under third effective oligomerization conditions to produce a third effluent, the third oligomerization catalyst comprising a crystalline molecular sieve. The third oligomerization catalyst may be the same as or different from the first and/or second oligomerization catalyst. It may be that, for example, the third effective oligomerization conditions comprise any feature described in relation to the first and/or second effective oligomerization conditions herein. The third effective oligomerization conditions may differ from, or be the same as, the first and/or second effective oligomerization conditions in the same way that the first effective oligomerization conditions differ from, or are the same as, the second effective oligomerization conditions. Optionally, the third effluent is contacted with a fourth oligomerization catalyst in a fourth reactor under fourth effective oligomerization conditions to produce a fourth effluent, the fourth oligomerization catalyst comprising a crystalline molecular sieve. The fourth oligomerization catalyst may be the same as or different from the first, second and/or third oligomerization catalyst. It may be that, for example, the fourth effective oligomerization conditions comprise any feature described in relation to the first, second and/or third effective oligomerization conditions herein. The fourth effective oligomerization conditions may differ from, or be the same as, the first, second and/or third effective oligomerization conditions in the same way that the first effective oligomerization conditions differ from, or are the same as, the second effective oligomerization conditions. Optionally, the first, second, third and fourth effluents, if present, comprise C₁₂ olefins. Preferably, the third effluent comprises a greater wt % C₁₂ olefins, such as at least 1 wt % more C₁₂ olefins, for example at least 2 wt % more C₁₂ olefins, based on the weight of the olefins in the third effluent, than the wt % C₁₂ olefins in at least one of the first and second effluents, based on the weight of the olefins in the first and second effluents. Preferably, the fourth effluent comprises a greater wt % C₁₂ olefins, such as at least 1 wt % more C₁₂ olefins, for example at least 2 wt % more C₁₂ olefins, based on the weight of the olefins in the fourth effluent, than the wt % C₁₂ olefins in at least one of the first, second and third effluents, based on the weight of the olefins in the first, second and third effluents. Optionally, the first oligomerization catalyst has been used in an oligomerization process for a first reaction period, the second oligomerization catalyst has been used in an oligomerization process for a second reaction period, the third oligomerization catalyst has been used in an oligomerization process for a third reaction period, and the fourth oligomerization catalyst (if present) has been used in an oligomerization process for a fourth reaction period, wherein the third and fourth (if present) reaction periods are different to at least one of the first and second reaction periods, optionally wherein the third and fourth (if present) reaction periods are longer than at least one of the first and second reaction periods. For example, it may be that the fourth reaction period (if present) is longer than the third reaction period, the third reaction period is longer than the second reaction period, and the second reaction period is longer than the first reaction period. In other words, the reactors comprise catalysts having different ages, the catalysts being arranged in age order with the newest catalyst being located in the first reactor and the oldest catalyst being located in the last reactor.

Additionally or alternatively, it may be that process comprises contacting the olefin feedstock the oligomerisation catalyst in a reaction zone comprising three or more reactors arranged in series. For example, it may be that the olefin feedstock is contacted with a first oligomerization catalyst under first effective oligomerization conditions in a first reactor of the three or more reactors. Optionally, in each reactor, the effluent from the previous reactor is contacted with a further oligomerization catalyst under further effective oligomerization conditions, the further oligomerization catalyst of each reactor being as described in relation to the oligomerization catalyst herein, and the further effective oligomerization conditions in each reactor being as described in relation to the effective oligomerization conditions herein. It may be that, for example, the oligomerization catalyst in each reactor is the same type of oligomerization catalyst (e.g. an intermediate pore size zeolite having a particular structure type). Preferably, the last reactor in the series of three or more reactors comprises the oldest of the oligomerisation catalysts in the reaction zone. For example, it may be that the reactors comprise catalysts arranged in ascending age order (e.g. the most fresh catalyst is located in the first reactor and the oldest catalyst is located in the last reactor of the three or more reactors). Preferably, the process is operated in a first process configuration and in a second process configuration as described in relation to the second aspect of the invention.

The present inventors have found that the use of multiple reactors allows the conditions of each reactor to be tailored to the catalyst in that reactor, for example tailored to the age and activity of the catalyst in that reactor.

Optionally, the process comprises contacting the olefin feedstock with the oligomerization catalyst in a reaction zone as described in relation to the second aspect of the invention.

Optionally, the process comprises analysing the oligomerization product using a gas chromatography-mass spectrometry analysis method. Preferably, the process comprises analysing the oligomerization product using the analysis method of the third aspect of the invention.

It may be that, in the olefin composition of the fourth aspect of the invention, the olefin composition comprising from 70 to 95 wt % C₁₂ olefin isomers, based on the weight of the olefin composition, the olefin composition comprising at least 50 mol % olefin isomers of type II and IVA, based on the moles of the olefin isomers in the olefin composition, the average branchiness of the olefin composition is in the range of from 2.6 to 3.3, optionally 2.6 to 2.95, for example 2.85 to 2.95. As used herein, olefin isomer types have the following meanings:

| Olefin | | Olefin |
|---|---|---|
| Type | Structure | Description |
| I | R1—C═C | α-Linear |
| II | R1—C═C—R2 | Internal Linear |
| III | 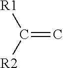 | α-Branched |
| IV | 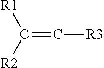 | Branched Internal |
| V | 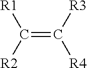 | Branched Internal |

In type IVA, one of R1, R2 or R3 is a methyl group.
In type IV B, R1, R2 and R3 are ethyl groups or larger.

Preferably, the olefin composition comprises at least 48 mol % olefin isomers of type IVA, based on the moles of the olefin isomers in the olefin composition. It may be that, for example, the olefin composition comprises at least 11 mol % olefin isomers having a carbon-carbon double bond in which each carbon of the carbon-carbon double bond is substituted by only one carbon atom, based on the moles of the olefin isomers in the olefin composition. Optionally, the olefin composition comprises at least 60 mol % olefin isomers of type IV, based on the moles of the olefin isomers in the olefin composition. Preferably, the olefin composition is produced by the process of the first aspect of the invention.

### EXAMPLES OF THE INVENTION

The following examples illustrate the present invention. Numerous modifications and variations are possible and it is to be understood that within the scope of the appended claims, the invention may be practiced otherwise than as specifically described herein.

### Example 1

Various C₆ olefin-containing feeds were subjected to an oligomerization process according to the first aspect of the invention using intermediate and large pore size catalysts. More particularly, C₆ olefin feeds from refinery/oligomerization plants. A typical composition of C₆ olefin feed is given in Table 1, the sulfur contents varying from 0 ppm to 26 ppm. It is believed that such amount does not affect the reaction and the properties of the oligomerisation product.

TABLE 1
Typical composition of C₆ olefin feed in percentages

| Component | Wt % |
|---|---|
| isobutane | 0.21 |
| n-Butane | 1.48 |
| isobutene | 0.00 |
| n-Butenes | 0.83 |
| C₄ dienes | 0.00 |
| C₄ cyclic hydrocarbons | 0.00 |
| (olefinic + saturated) | |
| isopentane + 2,2 di-methyl-propane | 1.36 |
| n-pentane | 0.08 |
| isopentenes | 4.12 |
| n-pentenes | 4.08 |
| C₅ dienes | 0.00 |
| C₅ cyclic hydrocarbons | 0.01 |
| (olefinic + saturated) | |
| Hexenes | 80.79 |
| C₆ saturated alkanes | 3.87 |
| C₆ cyclic hydrocarbons | 0.03 |
| (olefinic + saturated) | |
| C₆ dienes | 0.00 |
| Heptenes | 3.03 |
| C₇ saturated alkanes | 0.01 |
| C₇ cyclic hydrocarbons | 0.00 |
| (olefinic + saturated) | |
| C₇ dienes | 0.00 |
| >C₈ hydrocarbons | 0.03 |
| Total oxygenates (area %) | 0.06 |

Each C₆ olefin feed feed was contacted with the catalyst at temperatures of 150-230° C., a pressure of at 50 barg and a weight-hourly-space-velocity of 1-2 h⁻¹.

C₆ olefin dimerization product from a pilot plant run under the above conditions using a ZSM-57 zeolite catalyst showed a relatively high quaternary carbon content and unusual olefin type distribution compared to C₁₀-C₁₃ range oligomerization products produced from conventional C₃ or C₄ olefin feeds. It is believed that the observed differences in quaternary carbon content result from the different type of feedstock (i.e. C₆ olefin feedstock) used in the process rather than from the catalyst type.

A set of additional oligomerization runs (“Set A” runs) were carried out with different zeolite catalysts using C₆ olefin feeds and with variation of process temperature. The tested zeolite catalysts MCM-22, MCM-49, MCM-56, SAPO-11, ZSM-5, EMM-20, ZSM-11, ZSM-22, ZSM-23, ZSM-35, ZSM-48, ZSM-50 and ZSM-57, and large pore size, 12-membered ring zeolites Mordenite, Beta and Ultrastable Y (USY).

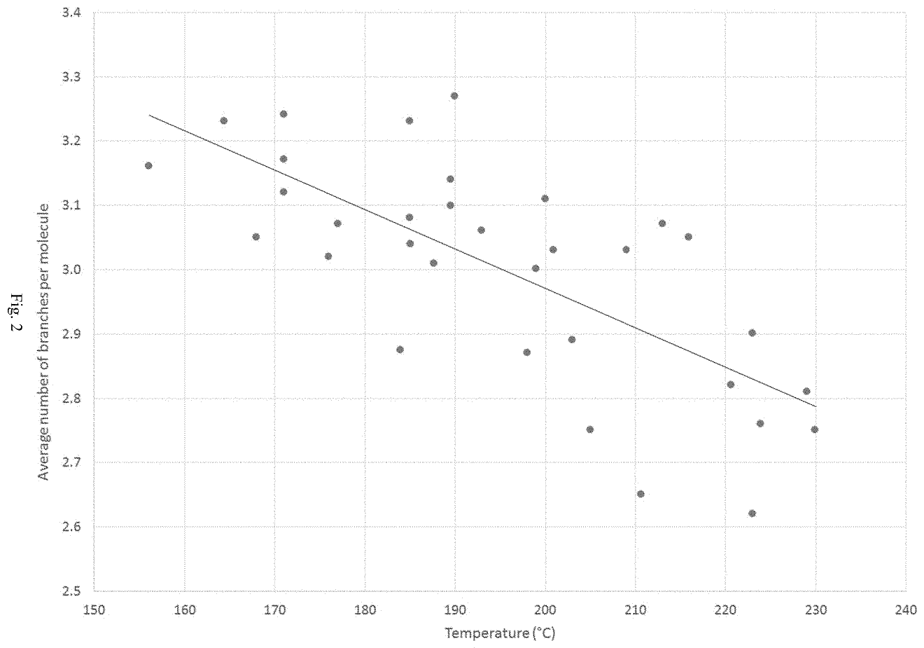

The branchiness of the oligomerization product produced in the Set A runs are set out in FIG. 2, which shows a graph plotting process temperature against oligomerization product branchiness (determined by NMR). The results set out in FIG. 2 show that process temperature can be used to control the degree of branching of the oligomerization product. The higher the temperature, the lower the branchiness. This trend was also confirmed using a GC-MS branchiness analyses according to the third aspect of the invention. Without wishing to be bound by theory, it is believed that lower branchiness at higher temperatures results from a combination of 1) the higher C₁₁-C₁₃ isomerization reactions at higher temperatures, 2) cracking to lighter olefins and recombination to C₁₁-C₁₃ range products at higher temperatures, and 3) higher conversion of more linear C₆ olefin isomers at higher temperatures. More particularly, it is believed that both isomerization and cracking would lead the composition to lower branchiness at higher temperature. Furthermore, the higher relative reactivity of linear C₆ olefins (as compared to branched C₆ olefins) in the oligomerization reaction also leads directly to a more linear C₁₂ range product. The results set out in FIG. 2 also show that the process of the present invention can be tuned by adjusting the temperature to provide oligomers having branchiness lower than 2.85. Thus, the process of the present invention provides both a route to less branched oligomers and also a flexible process in which branchiness can be tuned by varying process temperature.

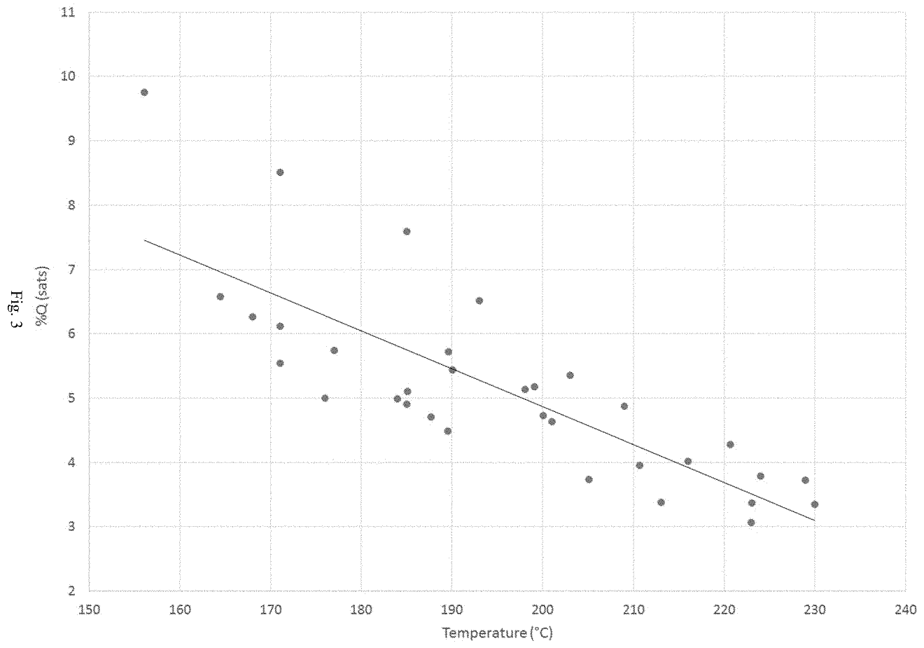

The quaternary carbon contents of the oligomerization product produced in the Set A runs are set out in FIG. 3, which shows a graph plotting process temperature against oligomerization product quaternary carbon content. Even though the nature of the catalyst seems to impact the quaternary carbon content, the results set out in FIG. 3 show that there is a clear effect of process temperature on quaternary carbon content. As it is believed that the presence of quaternary carbon atoms can reduce the activity of the oligomerization product in olefin-to-alcohol conversion (e.g. TDA synthesis), reduction in the amount of quaternary carbon is desirable.

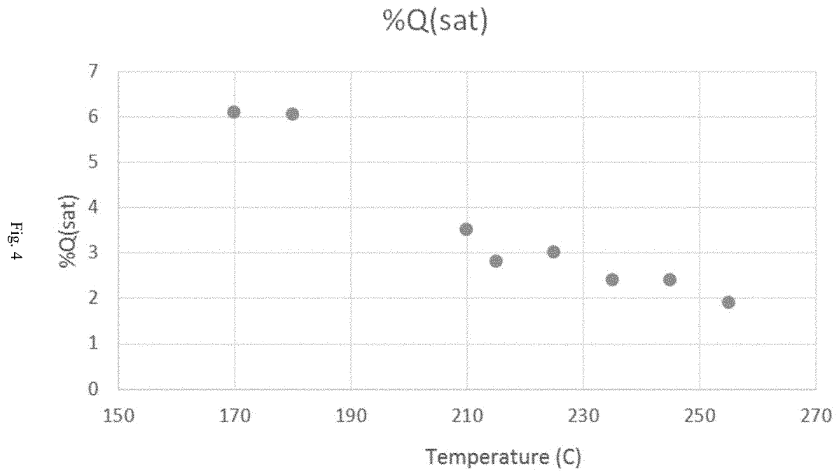

Further runs (“Set B” runs) were carried out to get a better understanding of the range of control of quaternary carbon content using process temperature. The results of the Set B runs set out in FIG. 4, which shows a further graph plotting process temperature against oligomerization product quaternary carbon content. The Set B results confirm the trend to lower quaternary carbon content with higher process temperature, and demonstrate that it is possible to reach average quaternary carbon content of lower than 2 wt % when the process temperature is increased.

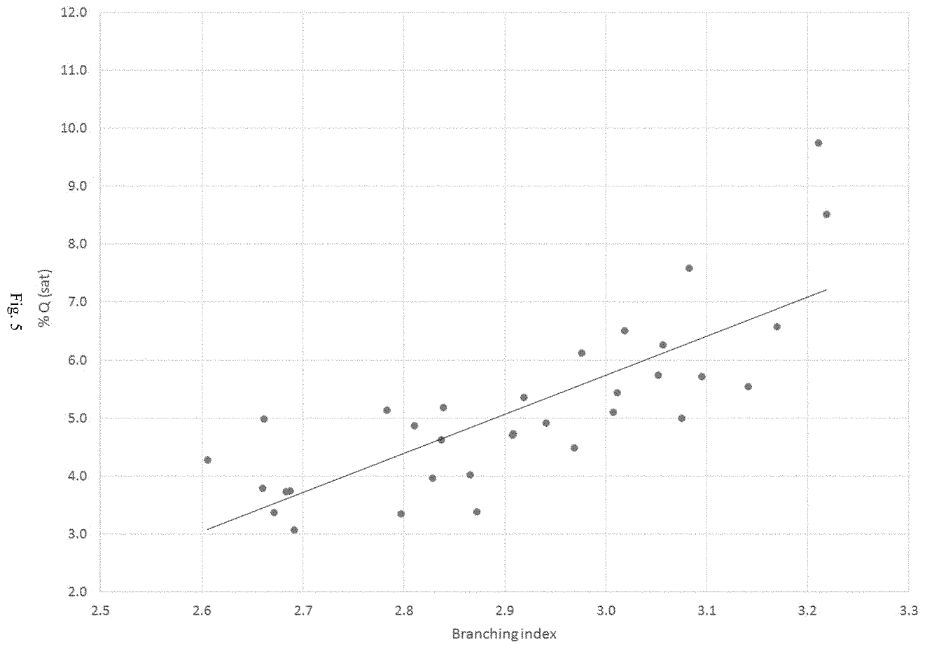

FIG. 5 shows a graph plotting oligomerization product quaternary carbon content against oligomerization product branchiness for the oligomerization products of the Set A runs. As shown in FIG. 5, there is a positive correlation between branchiness and quaternary carbon content in the oligomerization products.

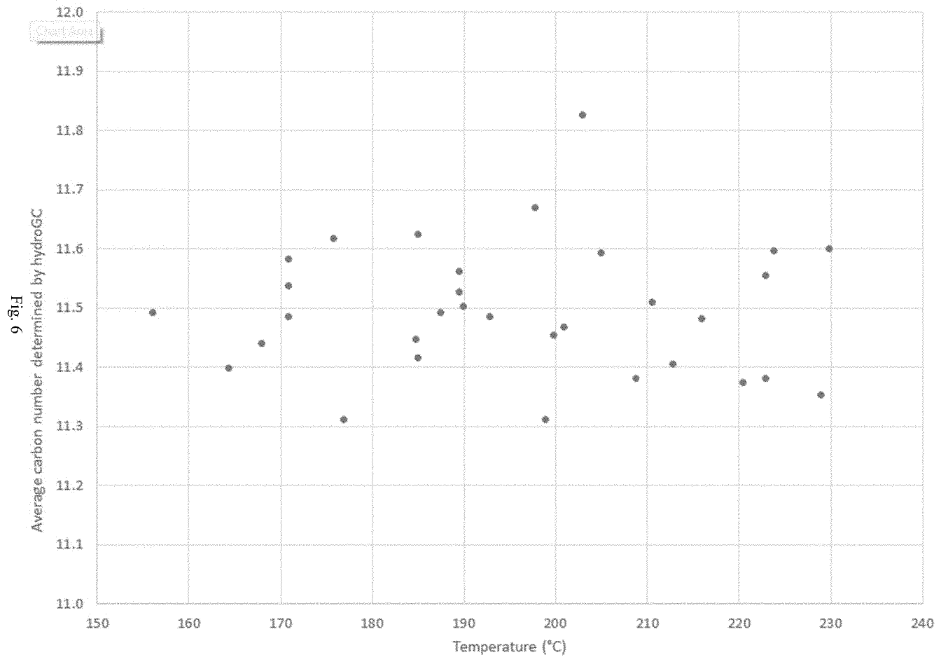

The oligomerization products of the Set A runs were further analysed to check for a relationship between process temperature and CND. The results of that analysis are set out in FIG. 6, which shows graph plotting process temperature against oligomerization product carbon number distribution. One graph plots CND as determined with hydro-GC. As shown in FIG. 6, there is no correlation between CND and temperature.

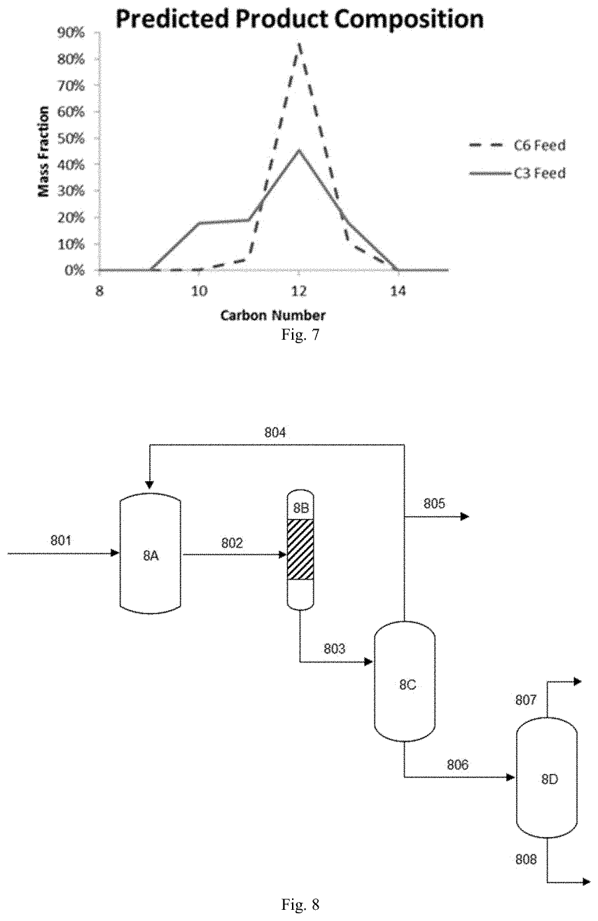

In order to provide a comparison between C₆-oligomer CND (i.e. an oligomerization product produced according to the present invention) and C₃-oligomer CND (i.e. a convention oligomerization product), a mathematical model was developed by fitting mathematical expressions to the experimental data from the Set A runs. The mathematical model is capable of predicting the performance of various feed, catalyst and processing conditions in various process configurations. The model was used to predict the product formed in a typical continuous commercial reaction process. The model was applied to C₃ olefin feeds and C₆ olefin feeds in order to illustrate the advantage of the new process over the traditional C₃ olefin oligomerization process. The results of the simulations are set out in FIG. 7, which shows a graph plotting carbon number against simulated mass fraction for an oligomerization product produced from a C₃ olefin feed and from a C₆ olefin feed. As shown in FIG. 7, the process utilizing a C₆ olefin feed is capable of producing a product with a much narrower distribution of molecule sizes that the process utilizing a C₃ olefin feed. Such greater control of the molecule size is advantageous because, for example, it allows for a certain amount of lower value product to be blended into the oligomerization product and still allow the blended product to meet established product specifications.

### Example 2

FIG. 8 shows a process schematic for an oligomerization process, the system comprising a feed vessel 8A, a reaction zone comprising a single reactor 8B, a separator 8C and a product purification column 8D. In this system, stream 801 is the fresh feed consisting of low molecular weight olefins and low reactivity, saturated hydrocarbons. Stream 802 is the total feed to the reaction zone which includes the fresh feed to the system and the recycle stream. Stream 803 is the raw product from the reaction zone containing olefin product, unreacted light olefins, low reactivity, saturated components, and heavy byproducts. Stream 804 is the recycle stream consisting of un-reacted olefins and low reactivity saturated components. Stream 805 is a purge stream intended to eliminate the buildup of low reactivity compounds in the system. Stream 807 is the desired oligomer product. Stream 808 is the unwanted heavy byproducts of the oligomerization reaction.

In oligomerization systems such as this, it is desirable to maximize the conversion of product into the oligomer product while minimizing heavy byproducts. The temperature, composition, flow rate, catalyst type, and amount of catalyst in the reaction zone are often fundamental parameters that control the selectivity in the reaction zone. Typically, a certain amount of unreacted olefin is lost in the saturate purge stream 805. Also, a certain amount of olefin feed is usually converted to heavy byproducts and lost in stream 808. The selection of several key parameters can dictate how efficiently these systems convert the olefin feed into the desired oligomer product. For example, it may be that the temperature in the reaction zone can be used to increase the conversion, and that the amount of catalyst in the reaction zone can also be used to increase the amount of feed that is converted in the reaction zone. In many systems, the size of the recycle stream 804 is also an important factor. In most cases there is an optimum per-pass conversion to maximize the product yield. For example, if the conversion is too low, it may be that more olefins will be lost to the purge stream, whereas if the conversion is too high, it may be that more olefins will be converted unwanted heavy byproducts.

A simulation has been developed to predict the yield from the system depicted in FIG. 8. This simulation incorporates both a model of the catalyst performance and a model of the process flow. The catalyst model predicts the reaction rate as a function of temperature and composition based on a fit to experimental data. The process flow model uses a mass and energy balance to calculate the flows into and out of each vessel shown in FIG. 8. This combined simulation can calculate the temperature and composition in the reaction zone and subsequently predict how these conditions affect the production rates. Overall, this simulation is capable of predicting the product yields as a function of recycle rate. When assessing the effectiveness of recycle rate as a tool to increase product yield, it is often important to hold certain parameters constant so that yields can be compared on a consistent basis. In the examples that follow the reactor temperature has been adjusted to fix the conversion in two ways. In Example 2a the total conversion is fixed, while in Example 2b the per-pass conversion is fixed.

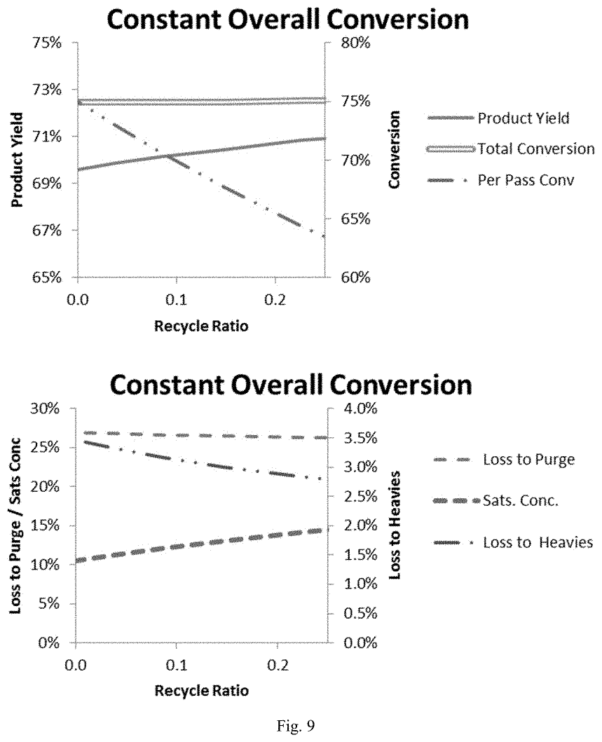

Example 2a—recycle rate is increased while adjusting the temperature to achieve a constant overall conversion (75%) of olefins. When the total flow rate of olefins into the reaction zone (stream 802) increases as the recycle rate (stream 804) is increased, and the total conversion is held constant based on the overall makeup (stream 801), the per pass conversion is decreased. In other words, the percentage of olefins actually entering the reactor that react into something else is reduced. Without wishing to be bound by theory, it is believed that the reduced per pass conversion reduces the amount of molecules in the reactor available for conversion into heavier byproducts. It is believed that, overall, this has the effect of reducing the olefins lost to heavy byproducts and increasing the overall product yield. The results of Example 2a are set out in FIG. 9, which shows a pair of graphs showing variation of product yield and per pass conversion with recycle ratio, and variation of loss to purge, saturates concentration and loss to heavies with recycle ratio, while overall conversion is kept constant.

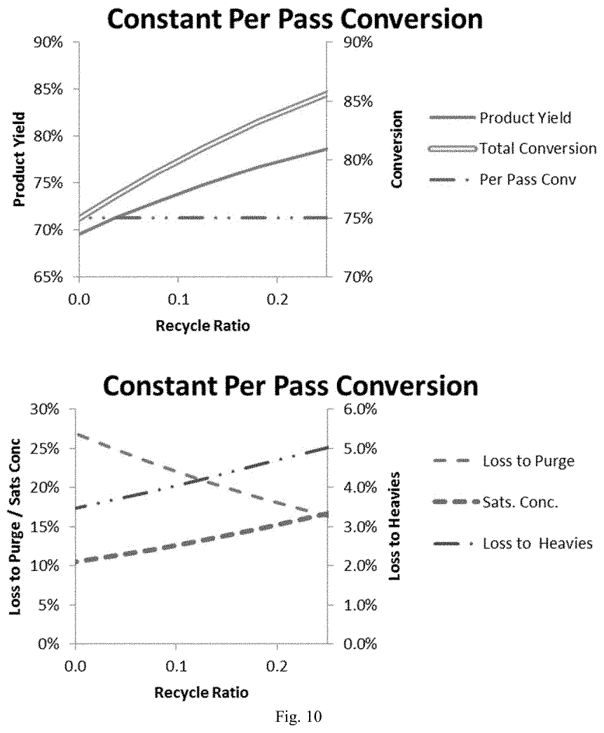

Example 2b—recycle rate is increased while adjusting the temperature to increase the conversion. In contrast to Example 2a, in Example 2b, the per-pass conversion (75%) is held constant rather than the total conversion. In other words, temperature in the reaction zone is increased to keep pace with the additional flow of olefins into the reaction zone even as that amount increases with additional recycle. It is believed that this operating mode nullifies the effect examined in Example 2a because, regardless of the number of olefins entering the reaction zone, the fraction that are reacted stays the same. However, even under these conditions the yield is improved as the recycle rate increases. In Example 2b, it appears that the concentration of unreactive saturated components has a strong effect on the yield. It is believed that, because the concentration of these components goes up with higher recycle rates, they serve to dilute the active components and reduce the loss of olefins in the purge stream. The results of Example 2b are set out in FIG. 10, which shows a pair of graphs showing variation of product yield and overall conversion with recycle ratio, and variation of loss to purge, saturates concentration and loss to heavies with recycle ratio, while per pass conversion is kept constant.

### Example 3

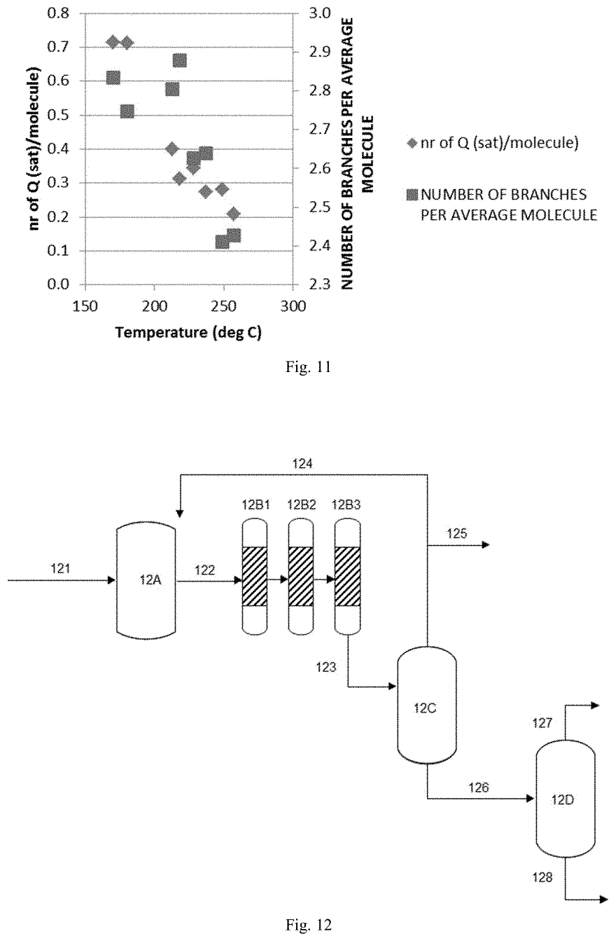

It has been found that, as a catalyst ages, reactor temperature often needs to be increased in order to maintain the same production rate. However, in addition to increasing the reaction rate, temperature can also affect the product quality in several ways. For example, it may be that higher reaction temperatures lead to more cracking of the reactor product and reduced product selectivity. For that reason, higher temperature can be undesirable in oligomerization reactions. Additionally or alternatively, temperature can have an effect on the isomer distribution in the product. For example, it may be that higher temperatures lead to fewer branches in the oligomer product and fewer quaternary carbons. Such an effect is set out in FIG. 11, which shows a graph plotting process temperature against oligomerization product quaternary carbon content and branchiness for an oligomerization product. Typically, reduced oligomerization product branchiness increase the reactivity of the oligomerization product in subsequent reactions, and reduced quaternary carbon content can increase the biodegradablity of the oligomerization product. For those reasons, higher temperature can be desirable in oligomerization reactions. It follows that changing reactor temperature as the catalyst ages can also lead to changes in product quality, which can itself be undesirable because it can make the product more or less suitable for certain applications.

It has been found that, by using multiple reactors, it is possible to distribute reactor temperature such that the total production rate is constant even as the catalyst ages in each reactor. Furthermore, it has been found that, by shifting more of the production from reactors containing old catalyst to reactors containing new catalyst over the course of the catalyst cycle, the outlet temperature of the final reactor can be maintained at a constant value. Maintaining a constant temperature in the final reactor in the series can help to maintain final product quality at a constant level, for example with regard to branchiness and quaternary carbon content, without increasing cracking in the upstream reactors.

In Example 3, a system was designed to test this this ability to improve product yield and quality through temperature control in multiple reactors. Such a system is depicted in FIG. 12, which shows a process schematic for an oligomerization process utilizing multiple reactors. The system of FIG. 12 consists of a total feed vessel 12A, a reaction zone with any number of multiple reactors 12B1, 12B2 and 12B3, a recycle column 12C and a product purification column 12D. In this system, stream 121 is the fresh feed consisting of low molecular weight olefins and low reactivity saturated hydrocarbons. Stream 122 is the total feed to the reaction zone which includes the fresh feed to the system and the recycle stream. Stream 123 is the raw product from the reaction zone containing olefin product, unreacted light olefins and low reactivity saturated components. Stream 124 is the recycle stream consisting of un-reacted olefins and low reactivity saturated components. Stream 125 is a purge stream intended to eliminate the buildup of low reactivity compounds in the system. Stream 127 is the desired oligomer product. Stream 128 is the unwanted heavy byproducts of the oligomerization reaction.

A simulation has been developed to predict the yield from the system depicted in FIG. 12. The simulation incorporates both a model of the catalyst performance and a model of the process flow. The catalyst model predicts the reaction rate as a function of temperature, catalyst age and composition based on a fit to experimental data. The process flow model uses a mass and energy balance to calculate the flows into and out of each vessel described in FIG. 12. This combined simulation can calculate the temperature and composition in the reaction zone and subsequently how these conditions affect the production rate. Furthermore, this model predicts how changes to the process configuration will affect the composition in the reaction zone and the ability to separate the product from the low reactivity saturates and heavy byproducts. The model does not predict the isomer distribution effects illustrated in FIG. 11, but it does establish reactor outlet temperature which will establish the isomer distributions as shown in FIG. 11.

Example 3a—control outlet temperature while maintaining constant yield. In this example, the catalyst in each reactor has a different age, as it would, for example, in a process in which a single reactor is periodically taken off-line to replace the catalyst. In this case, the freshest catalyst is in the first reactor 12B1. This example shows how the reactor temperature can be adjusted to maintain constant product yield and constant outlet temperature. In order to maintain a constant outlet temperature, more production is shifted to the front of the reaction train (fresher catalyst) as the catalyst ages. The ability to maintain constant yield and outlet temperature improves consistency in product quality with regard to branchiness and quaternary carbon content. Note that the outlet temperature of the last reactor in the reaction train, reactor 12B3, is constant for the duration of the cycle. The results of the simulation of Example 3a are set out in Table 2.

TABLE 2
Simulation Results for Example 3a

| | Olefins | | Reactor Inlet Temp. | | | Reactor Outlet Temp. | | | Days |
|---|---|---|---|---|---|---|---|---|---|
| Prod. | Lost to | Heavy | (° C.) | | | (° C.) | | | on |
| Yield | Purge | Yield | 12B1 | 12B2 | 12B3 | 12B1 | 12B2 | 12B3 | Stream |
| 82% | 11% | 7% | 154 | 195 | 242 | 163 | 204 | 249 | 0 |
| 82% | 11% | 7% | 162 | 207 | 244 | 173 | 217 | 249 | 45 |
| 82% | 11% | 7% | 170 | 217 | 246 | 181 | 228 | 249 | 90 |
| 82% | 11% | 7% | 176 | 226 | 247 | 188 | 238 | 249 | 135 |
| 82% | 11% | 7% | 184 | 238 | 248 | 196 | 250 | 249 | 179 |
| 82% | 11% | 7% | 191 | 247 | 248 | 203 | 259 | 249 | 224 |

Example 3b—improved yield with multiple reactors. In this example, all process conditions have been held constant except for the number of reactors. Here the product yield is improved by splitting the reaction over four reactors (12B1 to 12B4, 12B4 not shown in FIG. 12) with independent temperature control, rather than making all the product in one reactor. It is believed that the use of multiple reactors enables more precise temperature control and limits exposure of the reacting productions to undesirable temperatures. The yield improvement is illustrated both with and without recycle. The results of the simulation of Example 3b are set out in Table 3.

TABLE 3
Simulation Results for Example 3b

| | | Olefins | | | | | |
|---|---|---|---|---|---|---|---|
| Test | Product | Lost to | Heavy | Reactor Inlet Temp. (° C.) | | | |
| No. | Yield | Purge | Yield | 12B1 | 12B2 | 12B3 | 12B4 |
| 1 | 64.0% | 25.4% | 10.6% | 159 | 177 | 194 | 232 |
| 2 | 61.1% | 27.1% | 11.8% | 204 | n/a | n/a | n/a |
| 3 | 70.7% | 26.7% | 2.5% | 264 | 340 | 374 | 379 |
| 4 | 70.2% | 27.2% | 2.7% | 477 | n/a | n/a | n/a |

| | | |
|---|---|---|
| Test | Cumulative Olefin Conversion | Recycle |

| No. | 12B1 | 12B2 | 12B3 | 12B4 | Ratio |
|---|---|---|---|---|---|
| 1 | 50.0% | 69.9% | 79.9% | 84.9% | 0.00 |
| 2 | 84.9% | n/a | n/a | n/a | 0.00 |
| 3 | 40% | 60% | 70% | 75% | 1.08 |
| 4 | 75% | n/a | n/a | n/a | 1.08 |

Example 3c—improved yield with catalyst arrangement. This example shows how product yield is improved by arranging the oldest catalyst in the last reactor rather than arranging the reactors with the oldest catalyst in the lead reactor. All simulations were carried out with four reactors (12B1 to 12B4, 12B4 not shown in FIG. 12). In tests 1 and 2, the reactors are arranged so that the oldest catalyst is in the last reactor. In tests 3 and 4, the oldest catalyst is in the lead reactor. This principle is illustrated both with and without recycle. The results of the simulation of Example 3c are set out in Table 4.

TABLE 4
Simulation Results for Example 3c

| | | Olefins | | | | | | |
|---|---|---|---|---|---|---|---|---|
| Test | Product | Lost to | Heavy | Cumulative Olefin Conversion | | | | Recycle |
| No. | Yield | Purge | Yield | 12B1 | 12B2 | 12B3 | 12B4 | Ratio |
| 1 | 64.0% | 25.4% | 10.6% | 50% | 70% | 80% | 85% | 0.00 |
| 2 | 61.9% | 26.6% | 11.5% | 5% | 15% | 35% | 85% | 0.00 |
| 3 | 78.1% | 17.2% | 4.6% | 50% | 70% | 80% | 85% | 1.15 |
| 4 | 77.9% | 17.4% | 4.7% | 5% | 15% | 35% | 85% | 1.14 |

### Example 4

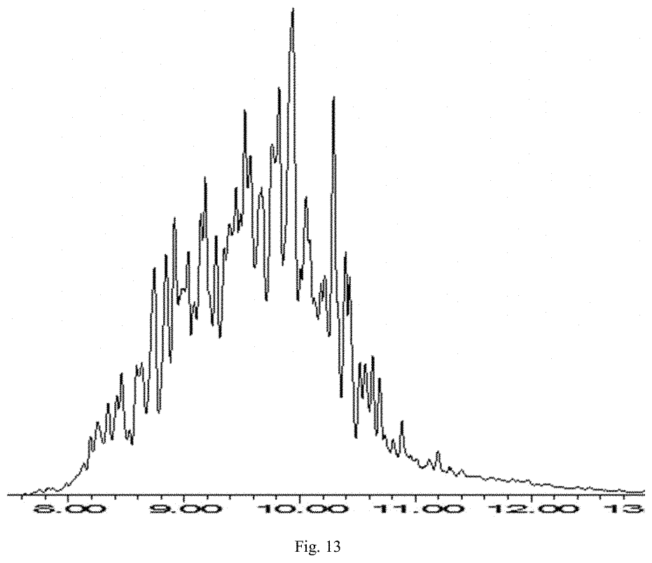

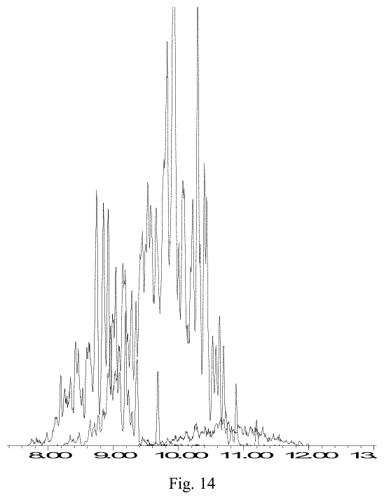

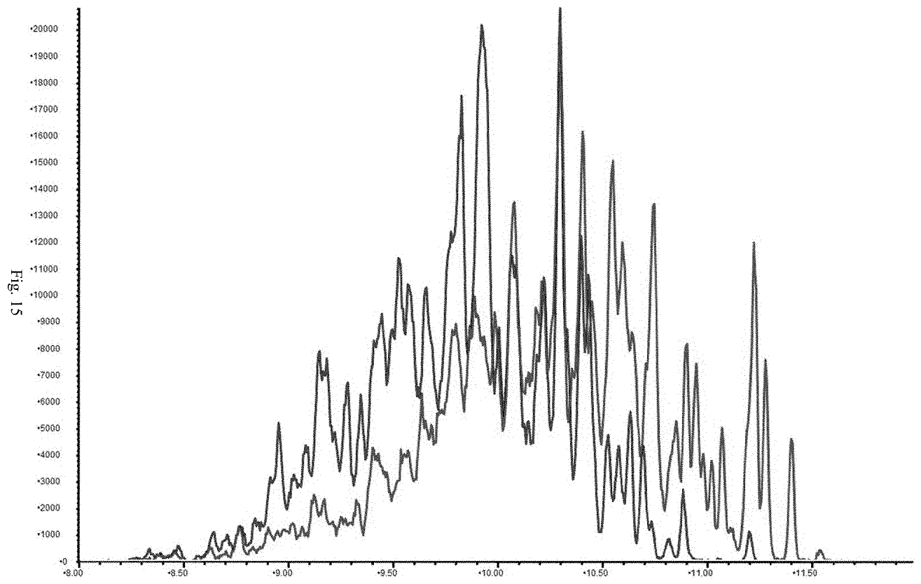

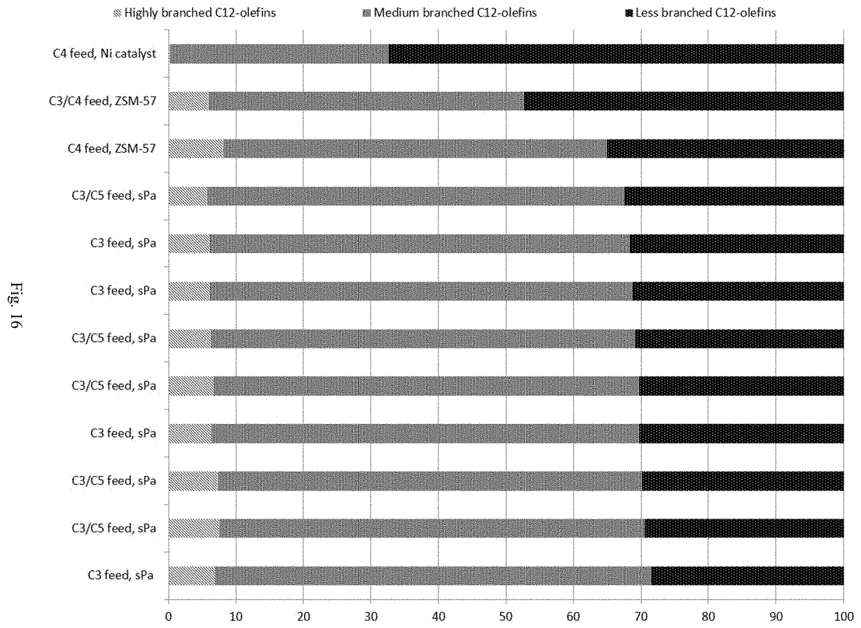

It has been found that a combination of chromatography and mass spectrometry can provide an insight into the isomer mixture/branchiness of a particular Cₙ olefin in a mixture of Cₙ₋₁, Cₙ and Cₙ₊₁ olefins. In particular, a gas chromatography-mass spectrometry (GC-MS) method, primarily targeting C₁₂ olefins, was developed. Rather than looking at the complete isomer mixture present in the sample, the method focused on C₁₂ olefins only. It has been found that, by doing so, chemical differences (i.e. level of branchiness of the available isomers) introduced by heart-cutting, for example, were eliminated. Differences between the C₁₂ olefin extracted ion chromatograms of different samples was captured using a visualization model. This approach allowed the ranking of a large variety of plant samples and research samples as a function of their branchiness. The ranking order of these samples can be used, for example, to predict the suitability of different C₁₂-range products for certain applications. For instance, products containing a highest amount of “more linear” C₁₂ olefins may be more suitable for conversion to C₁₃-alcohols than those containing a lower amount of more linear C₁₂ olefins. An apolar capillary gas chromatography (GC) column was used to separate the components present in the isomeric mixture based on their boiling point. The mass spectrometer was used in full scan mode, with a scan range from 35.0 to 280 m/z, to record the chromatogram shown in FIG. 13. This complex mixture containing 2000+ C₁₂ olefin isomers and also some C₁₁ and C₁₃ olefins cannot be fully resolved. In order to visualize the C₁₁, C₁₂ and C₁₃ olefins, their respective molecular (or parental) ions (154, 168 and 182 m/z) were extracted from the total ion chromatogram. An overlay of the three extracted ion chromatograms is shown in FIG. 14. High boiling C₁₁ olefins overlap with low boiling C₁₂ olefins, whereas high boiling C₁₂ olefins overlap with low boiling C₁₃ olefins. In order to calculate the GC-MS derived C₁₂ olefin branching index, special attention was paid to the C₁₂-fraction. An overlay of the extracted ion chromatogram (m/z 168) of two different C₁₂-range olefin samples is shown in FIG. 15, which indicates that the two extracted ion chromatograms clearly differ from each other. To capture the differences, a visualization method was developed.

Heptane, 2, 2, 6, 6-tetramethyl-4-methylene and 1-dodecene were used to define the start and end point of the C₁₂ olefin retention time zone. The HP-5MS column used for the GC separation separates compounds based on their boiling point, and so compounds with the lowest retention time were assumed to be highly branched (lowest boiling point) and the compounds with the highest retention time were assumed to be less branched (highest boiling point). The C₁₂ olefin fraction was partitioned into three equally sized parts, which were defined as groups of highly, medium and less branched C₁₂ olefin isomers. The response to ion m/z-168 was summed up for each of the three zones and divided by the total response to ion m/z-168 to calculate the relative response of the three fractions with respect to the total C₁₂-fraction. It has been found that, by plotting these normalized numbers for the highly, medium and less branched C₁₂ olefin isomers, the relative contribution of the less branched fraction with respect to the total C₁₂-fraction between different samples can be readily compared. This analytical method was used to measure the C₁₂ olefin branching index of both heart-cut distillated plant samples and research samples containing a lower concentration of higher molecular weight olefins. Instrumentation details for the GC-MS measurement method are set out in Table 5.

TABLE 5
Instrumental Details for the GC-MS branchiness analysis method
GC-parameters

| GC-type | Agilent 7890A | |
|---|---|---|
| Column | HP-5MS, 30 m × 0.25 mm ID, dft 0.25 μm | |
| Carrier gas | Helium | |
| Flow | 0.88852 | ml/min* |
| Pressure | 13.234 | psi* |

Inlet-settings

| Inlet temperature | 250° | C. |
|---|---|---|
| Mode | split | |
| Split ratio | 1/300 | |
| Injection volume | 0.5 | μl |

Oven Method

| | | |
|---|---|---|
| Initial temperature | 50° | C. |
| Initial time | 2 | min |
| Rate | 10° | C./min |
| Final temperature | 310° | C. |
| Hold time | 2 | min |

MS parameters

| MS-type | Agilent 5975C | |
|---|---|---|
| Temp transfer line | 280° | C. |
| Tune file | atune.u | |
| Solvent delay | 3 | min |

| | |
|---|---|
| EMV mode | Gain factor |
| Gain factor | 0.5 |
| | 100 |
| Acquisition mode | Scan |

Scan parameters

| Mass range | 35-280 | |
|---|---|---|
| Threshold | 100 | |
| MS source temperature | 230° | C. |
| MS quad temperature | 150° | C. |

*determined by retention time locking on n-C16 peak

The retention times of Heptane, 2, 2, 6, 6-tetramethyl-4-methylene and 1-dodecene were found to be 8.304 and 11.276 minutes, respectively. These retention times are defined experimentally for each analysis, by injecting the pure components, in case a significant retention time shift of n-C₁₆ is observed. The retention times might shift depending on instrument used and column age. Retention time locking can be used to prevent need for adjustment of retention time windows.

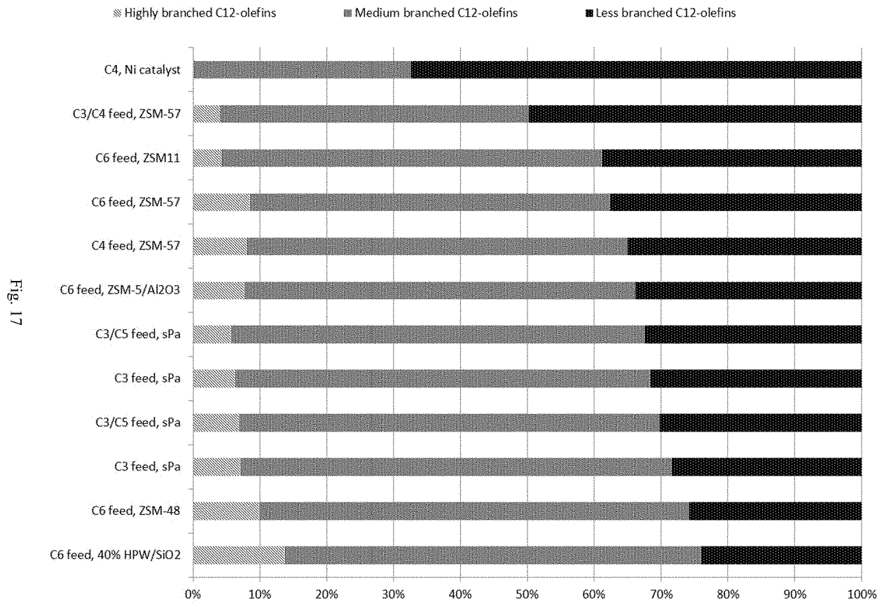

Example 4a—analysis of various C₁₂ olefin-containing mixtures, including heart-cut distillation plant samples. The results of the analysis of Example 4a are set out in FIG. 16, in which various C₁₂-range olefin samples are ranked according to the relative amount of “less branched C₁₂ olefins”. It has been found that this approach allows differentiation between C12 olefin mixtures prepared by conventional C₃/C₄ olefin oligomerization processes (“tetramer”) and other C₁₂-based higher olefins.

Example 4b—analysis of non-distilled oligomerization products. The results of the analysis of Example 4b are set out in FIG. 17, in which various C₁₂-range olefin samples are ranked according to the relative amount of “less branched C₁₂ olefins”. Example 4b shows that the analysis method can also be applied to characterize non-distilled oligomerization products containing a low amount of C₁₂ olefins. It follows that the analysis method allows the comparison of commercially available C₁₂-range samples with research samples.

### Example 5

In order to show the differences between C₁₂ oligomerization products prepared according to the process of the first aspect of the invention (inventive C₁₂ oligomerization product) and C₁₂ oligomerization products prepared by a conventional C₃/C₄ oligomerization process (comparative C₁₂ oligomerization product). The inventive and comparative oligomerization products were subjected to analysis by NMR. The results of that NMR analysis are set out in Table 6 below.

TABLE 6
NMR analysis of an Inventive C₁₂ oligomerization
product a Comparative C₁₂ oligomerization product

| | Comparative C₁₂ | Inventive C₁₂ |
|---|---|---|
| | oligomerization | oligomerization |
| | product | product |
| CARBON NUMBER | 11.77 | 11.89 |
| % SATURATES (GC) | 0.33 | 0.16 |
| If not measured, | | |
| value set to 0 | | |

OLEFIN TYPES

| | | |
|---|---|---|
| TYPE I | 1.34 | 0.28 |
| TYPE II | 10.31 | 11.67 |
| TYPE III | 6.95 | 5.37 |
| TYPE IV A | 44.77 | 49.37 |
| TYPE IV B | 14.52 | 13.10 |
| TYPE V | 22.11 | 20.21 |
| sum reactive | 63.37 | 66.68 |
| sum unreactive | 36.63 | 33.32 |

NUMBER OF CARBONS PER AVERAGE MOLECULE

| | | |
|---|---|---|
| CH₃ | 3.69 | 3.61 |
| CH₂ | 2.38 | 2.58 |
| CH | 0.38 | 0.33 |
| C | 0.32 | 0.35 |
| CH₃ ON OLEFIN | 1.21 | 1.21 |
| CH₂ ON OLEFIN | 1.16 | 1.29 |
| CH ON OLEFIN | 0.42 | 0.28 |
| C ON OLEFIN | 0.22 | 0.24 |
| CH₂═ | 0.08 | 0.06 |
| CH═ | 0.81 | 0.86 |
| C═ | 1.10 | 1.08 |
| TOTAL | 11.77 | 11.89 |

NUMBER OF BRANCHES PER AVERAGE MOLECULE

| | | |
|---|---|---|
| TOTAL | 2.98 | 2.87 |

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. A process for oligomerizing an olefin feedstock to form an oligomerization product, wherein the process comprises contacting the olefin feedstock with an oligomerization catalyst under effective oligomerization conditions;
wherein, the olefin feedstock comprises at least 50 wt % of one or more C₆ olefins, based on the weight of the olefins in the olefin feedstock;
and wherein, the oligomerization catalyst comprises a crystalline molecular sieve.

2. The process according to claim 1, wherein the crystalline molecular sieve comprises at least one of an intermediate pore size crystalline molecular sieve having 10-membered ring pores, or a large pore size crystalline molecular sieve having 12-membered ring pores.

3. The process according to claim 2, wherein the intermediate pore size crystalline molecular sieve, if present, is a zeolite having a structure type selected from the list consisting of AEL, MFI, MFS, MEL, MRE, MTW, MWW, EUO, MTT, HEU, FER, and TON, and the large pore size crystalline molecular sieve, if present, is a zeolite having a structure type selected from the list consisting of LTL, VFI, MAZ, MEI, FAU, EMT, OFF, BEA, and MOR.

4. The process according to claim 1, wherein the olefin feedstock comprises at least 60 wt %, of one or more C₆ olefins based on the weight of the olefins in the olefin feedstock, optionally wherein the olefin feedstock comprises at least 55 wt % of one or more C₆ olefins based on the weight of the olefin feedstock.

5. The process according to claim 1, wherein the oligomerization product comprises C₁₂ olefins.

6. The process according to claim 1, wherein the effective oligomerization conditions include at least one of: (i) a temperature of from 100° C. to 330° C.; (ii) a pressure of from 3 MPa to 10 MPa; and a weight hourly space velocity from 0.1 to 20 h⁻¹.

7. The process according to claim 1, wherein the process comprises separating the oligomerization product into a recycle stream and a further processing stream, the recycle stream comprising olefins of carbon number less than 12 and the further processing stream comprising oligomers;
and wherein the process comprises contacting the olefin feedstock with the oligomerization catalyst under the effective oligomerization conditions in the presence of the recycle stream;
and optionally separating the further processing stream into a product stream and a heavies stream, the product stream comprising oligomers, and the heavies stream comprising heavy by-products; optionally wherein the process comprises further separating a purge stream from the recycle stream, the purge stream comprising low reactivity by-products.

8. The process according to claim 7, comprising:
operating the process in a first process configuration in which the recycle stream is recycled at a first recycle flow rate, the olefin feedstock is contacted with the oligomerization catalyst at a first temperature, and olefins in the olefin feedstock are converted to oligomers in the further processing stream at a first conversion rate; and,
operating the process in a second process configuration in which the recycle stream is recycled at a second recycle flow rate, the olefin feedstock is contacted with the oligomerization catalyst at a second temperature, and olefins in the olefin feedstock are converted to oligomers in the further processing stream at a second conversion rate;
wherein the second recycle flow rate is greater than the first recycle flow rate, and wherein the first temperature and the second temperature are selected such that the first conversion rate is substantially the same as the second conversion rate, optionally wherein the first conversion rate and the second conversion rate are about 75%.

9. The process according to claim 7, comprising:
operating the process in a first process configuration in which the recycle stream is recycled at a first recycle flow rate, the olefin feedstock is contacted with the oligomerization catalyst at a first temperature, and olefins comprising: a) olefins in the olefin feedstock, and b) olefins in the recycle stream, are converted to oligomers in the further processing stream at a first conversion rate; and,
operating the process in a second process configuration in which the recycle stream is recycled at a second recycle flow rate, the olefin feedstock is contacted with the oligomerization catalyst at a second temperature, and olefins comprising: a) olefins in the olefin feedstock, and b) olefins in the recycle stream, are converted to oligomers in the further processing stream at a second conversion rate;
wherein the second recycle flow rate is greater than the first recycle flow rate, and wherein the first temperature and the second temperature are selected such that the first conversion rate is substantially the same as the second conversion rate, optionally wherein the first conversion rate and the second conversion rate are about 75%.

10. The process according to claim 1, wherein the olefin feedstock is contacted with the oligomerisation catalyst in a reaction zone comprising three or more reactors arranged in series, wherein the olefin feedstock is contacted with a first oligomerization catalyst under first effective oligomerization conditions in a first reactor of the three or more reactors, wherein, in each subsequent reactor in the series of three or more reactors, the effluent from the previous reactor is contacted with a further oligomerization catalyst under further effective oligomerization conditions, and wherein the last reactor in the series of three or more reactors comprises the oldest of the oligomerisation catalysts in the reaction zone.

11. The process according to claim 10, wherein the process is operated in a first configuration for a first operating period and subsequently in a second configuration for a second operating period,
wherein, the outlet temperature of the last reactor in the series of three or more reactors is substantially the same in the first and second configurations;
and wherein, in the second configuration, at least one of:
the inlet and/or outlet temperature of at least one of the reactors other than the last reactor, or
the inlet temperature of the last reactor,
differs from the corresponding inlet and/or outlet temperature of that reactor in the first configuration.

12. The process according to claim 1,
wherein a major portion of the olefin feedstock is a stream recovered from:
a light olefin oligomerization process;
a thermal hydrocarbon conversion process;
a heavy hydrocarbon catalytic conversion process;
a methanol catalytic conversion process;
and/or
a syngas catalytic conversion process;
optionally wherein the stream is recovered by distillation, adsorption, extraction, and/or membrane separation.

13. The process according to claim 1,
wherein the process comprises subjecting at least a portion of the oligomerization product to a gas chromatography-mass spectrometry analysis method, the analysis method comprising:
selecting a molecular ion, such as a C₁₂ molecular ion, for mass spectrometry detection;
selecting a gas chromatography start point and a gas chromatography end point to define a gas chromatography retention time zone extending from the start point to the end point;
dividing the gas chromatography retention time zone into a plurality of sections, each section corresponding to a group of molecular ion isomers; and,
determining total detection of the molecular ion in each of the plurality of retention time zone sections thereby determining the relative amounts of each group of molecular ion isomers.

14. The process according to claim 13, wherein the gas chromatography start point corresponds to the retention time of a highly branched isomer of the molecular ion, and wherein the gas chromatography end point corresponds to the retention time of a substantially linear isomer of the molecular ion.

15. An olefin composition comprising from 70 to 95 wt % C12 olefin isomers, based on the weight of the olefin composition, wherein the olefin composition comprises at least 50 mol % olefin isomers of type II and IVA, based on the moles of the olefin isomers in the olefin composition, and wherein the average branchiness of the olefin composition is in the range of from 2.6 to 3.3, optionally 2.6 to 2.95.

16. An olefin composition having an initial boiling point of 185° C. and a final boiling point of 210° C. and comprising from 70 wt % to 95 wt % C12 olefin isomers, from 8 wt % to 20 wt % C11 olefins, and from 1 wt % to 12 wt % C13 olefins, based on the weight of the olefin composition.

17. (canceled)

18. The process according to claim 1, wherein the crystalline molecular sieve comprises at least one of an intermediate pore size crystalline molecular sieve or a large pore size crystalline molecular sieve.

19. The process according to claim 3, wherein the intermediate pore size crystalline molecular sieve, if present, is a zeolite selected from the list consisting of MCM-22, MCM-49, MCM-56, SAPO-11, ZSM-5, EMM-20, ZSM-11, ZSM-22, ZSM-23, ZSM-35, ZSM-48, ZSM-50 and ZSM-57, and the large pore size crystalline molecular sieve, if present, is a zeolite selected from the list consisting of Mordenite, Beta and Ultrastable Y (USY).

20. The process according to claim 5, wherein the oligomerization product comprises at least 60 wt % C₁₂ olefins.
